(19) United States
(12) Patent Application Publication
(10) Pub. No.: US 2026/0083560 A1
(43) Pub. Date: Mar. 26, 2026

(54) ANNULOPLASTY METHODS AND DEVICES, SYSTEMS AND METHODS FOR THE DELIVERY OF AN ANNULOPLASTY DEVICE

(71) Applicant: Cinch Medical AS, Bergen (NO)

(72) Inventors: Philip Jon Haarstad, Chanhassen, MN (US); Lucas Harder, Minneapolis, MN (US)

(73) Assignee: Cinch Medical AS, Bergen (NO)

(21) Appl. No.: 18/893,811

(22) Filed: Sep. 23, 2024

(51) Int. Cl. A61F 2/24

(52) U.S. Cl. CPC A61F 2/2445; A61F 2/2466; A61F 2220/0016; A61F 2250/0004

(57) ABSTRACT

The present invention relates to a device for the delivery and securing of an intracorporeal device to a target site in a patient, wherein the delivery device comprises a shaft and a plurality of arms coupled to said shaft and configured to expand radially outwards relative to said shaft, wherein each arm comprises first means for coupling with the intracorporeal device and second means for releasing the intracorporeal device.

The present invention relates to heart valve repair procedures and devices, and more particularly to transcatheter annuloplasty procedures and devices. The present invention further relates to systems and methods for the delivery of a medical device, such as a medical implant and more particularly of an annuloplasty device.

### BACKGROUND OF THE INVENTION

Annuloplasty is a procedure for the repair of a heart valve, by tightening or strengthening the annulus around a valve. Annuloplasty procedures are often used to treat heart valve regurgitation. By way of example, the mitral valve regulates blood flow from the left atrium to the left ventricle, and prevents blood from flowing back into the left atrium from the left ventricle. Mitral valve regurgitation is a condition which occurs when blood flows backwards into the left atrium across a so-called “leaky heart valve”. This backflow is caused by a dysfunctional heart valve, which may be dysfunctional through injury, malformation and/or disease.

If the condition is minor, little or no treatment is required. However, in some cases, the blood backflow places a strain on the heart and causes it to work harder to compensate for the leaked volumes. A conventional and effective surgical method for treating this condition is with the use of an annuloplasty band or ring, which reduces the size of the annulus around the leaky valve to restore normal working function.

In recent years, alternatives procedures, in particular using transcatheter technologies, have been developed to treat patients for whom surgical intervention is unsuitable. Transcatheter or percutaneous procedures are minimally invasive, and hence minimally traumatic.

A major challenge in transcatheter procedures is accessibility, including the delivery of medical implants, tools and devices to the target site, and their precise and safe manipulation at the target site. Despite considerable advances in this field, it remains difficult to implant miniaturised devices in areas hindered by the patient's anatomy. Moreover, the medical practitioner must consider the specific anatomical landscape such as shape, dimensions, damage, and condition) of the target area for a successful implantation.

Whilst in surgical procedures, an annuloplasty device of suitable shape and size can be directly manipulated, positioned, and attached to the dysfunctional annulus, transcatheter implantation requires imaging equipment to visualise the device delivery path and the target site. There is also added complexity due to the accessibility of the target area, lack of direct visualisation and reduced size of the transcatheter tools and devices.

In transcatheter procedures, the annuloplasty device is delivered (and the medical tools manipulated) through a catheter, which conventionally travels through the patient's circulatory system. Due to the size restrictions, it is therefore difficult, if not impossible, to deliver an annuloplasty device with a fixed configuration. The fixed configuration would be the configuration of the device required to restore the annulus to a working configuration, and the lumen of the delivery catheter would need to be sufficiently large not only to accommodate this final configuration, but also to provide unhindered space for the surgeons to manipulates the tools and devices. Such a large catheter cannot travel through the patient's circulatory system. As a result, transcatheter annuloplasty devices are contractible or collapsible to enable delivery through a catheter.

Most established transcatheter annuloplasty procedures involve the use of a flexible or semi-flexible band. Once at the target area, the band is positioned by the surgeon, sutured, screwed, or otherwise anchored to the degenerated annulus to form the desired final working configuration, and subsequently tightened to narrow the annulus.

Thus, it is a requirement for transcatheter annuloplasty devices to be collapsible in order to be delivered through a catheter, and a consequence of this requirement is that the surgeon must reconstruct in situ the working configuration of the annuloplasty device.

The heart valve anatomy is complex in itself, and repairing a heart valve with the currently available techniques is equally so. The development of new annuloplasty devices and of corresponding delivery tools, is costly, and medical practitioners are required to undertake extensive and intensive training specific to each device-type before being able to fit them into patients. The process of establishing each new device in current medical practice is therefore highly resource-intensive.

Moreover, even with sophisticated devices and tools, intensive training and expert surgical intervention, there are so many variables at play that the outcome of the procedure is unpredictable. It is difficult to predict whether the band has been suitably positioned and anchored so that regurgitation may be significantly minimised, until the band has been tightened. At this stage, the device has already been sutured to the annulus, and can only be retrieved through surgery, if at all. It is also difficult to predict if the restoration of the valve is durable. In addition, repeated attempts at successful implantation and trial and error is damaging and traumatic to the patient's anatomy. Despite all efforts, the band may still fail to successfully restore the valve.

It is an object of this invention to mitigate problems such as those described above. In particular, there is a need for a simpler, more standardised, less resource-intensive and less traumatic system and method for repairing leaky heart valves. There is further a need for a system and method for the easy manipulation, efficient delivery, and accurate positioning of an annuloplasty device. There is also the need for a system and method which enable the repositioning of an annuloplasty device wherever required.

WO 2022/157225, the content of which is incorporated herein by reference, describes an annuloplasty device for restoring a heart valve annulus from a dysfunctional configuration to a working configuration. The device comprises a band or ring which is expandable from a pre-set configuration corresponding to a working configuration of the annulus. Upon release, the device reverts to its pre-set configuration thereby automatically contracting the annulus into its working configuration. WO 2024/013140, the content of which is incorporated herein by reference, describes a system for delivering the annuloplasty device of WO 2022/157225. The delivery device comprises a structure configured to expand from a first configuration to a second configuration, the dimensions of which are substantially the same or greater than the dimensions of the pre-set configuration of the annuloplasty device.

It is another object of this invention to provide improved alternatives to existing products. In particular, it is an object of the invention to improve patient safety during the delivery and implantation process. It is another object the invention to minimize the risk of injury to the patient during the delivery and implantation process. It is an other object of the invention to improve the anchoring of the implant to the patient's tissues and to minimize the risk of the implant becoming accidentally detached.

### STATEMENT OF INVENTION

The present disclosure concerns a new intracorporeal implant, a new delivery device and a new system including the implant and the delivery device. The implant includes, amongst other features, features which ensure safe and efficient anchoring to the target site in the patient. The delivery device includes, amongst other features, features which ensure the safe delivery of the implant to the target site in the patient. The delivery device is also particularly suited to the delivery, positioning and manipulation of the implant for optimum implantation. The implant and delivery device are each configured for optimum attachment, movement, and detachment.

According to an aspect of the present disclosure, there is provided a device for the delivery and securing of an intracorporeal device to a target site in a patient, wherein the delivery device comprises a shaft and a plurality of arms coupled to said shaft and configured to expand radially outwards relative to said shaft, wherein each arm comprises first means for coupling with the intracorporeal device and second means for releasing the intracorporeal device.

As used herein, the term “means” can be equivalently expressed as, or substituted with, any of the following terms: device, apparatus, structure, part, sub-part, component, assembly, sub-assembly, machine, mechanism, article, medium, material, appliance, equipment, system, body or similar wording.

The present delivery device ideally comprises a first means for coupling with the intracorporeal device and second means for releasing the intracorporeal device. First and second means may be integrated into a connector. The separation of the two functions (coupling and releasing) allows for greater safety and control of the delivery and implantation process. Minimal modifications are required to the intracorporeal implant device. In fact, the design of the intracorporeal implant device may be simplified, so that the delivery device (or the multi-functional connector of the delivery device) interacts with the same portion of the intracorporeal implant device to couple the implant to the delivery device, to position the implant relative to the target site, to adjust the configuration of the implant and/of the anchoring means, and to release the implant from the delivery device.

Each arm, a group of arms, all the arms may comprise first coupling means. Each first coupling means and component of first coupling means may be adjusted and controlled individually or as a group. Each arm, a group of arms, all the arms may comprise second releasing means. Each second releasing means and component of second releasing may be adjusted and controlled individually or as a group.

In embodiments, first means for coupling with the intracorporeal device comprises a paddle having at least one recess configured to receive an attachment portion of the intracorporeal device.

Within the context of the present disclosure, a paddle designates or includes a component with substantially flat surface. The recess may be formed in the substantially flat surface of the paddle. The paddle may or may not extend from a pole coupled to an arm or directly from an arm of the delivery device.

In embodiments, the attachment portion of the intracorporeal device comprises or consists of a crest, preferably part of a crest, more preferably the distal portion of a crest of an intracorporeal implant device. In embodiments, the attachment portion of the intracorporeal device comprises or consists of a crest, preferably part of a crest, more preferably the distal portion of a crest of an intracorporeal implant device, and an anchoring means, preferably part of an anchoring means, more preferably the portion of the anchoring means connected to the crest.

In embodiments, the anchoring portion of the anchoring means is not received in the recess. That is, the anchoring portion may extend beyond the recess. The anchoring portion is the portion or end portion of the anchoring means which penetrates the patient's tissues to secure the intracorporeal implant device to the patient's tissues.

In embodiments, the recess is configured to couple with the intracorporeal device by friction. In embodiments, the recess is shaped and dimensioned to receive the attachment portion of the intracorporeal device. In embodiments, the recess may have an inner contour which is substantially the same as the outer contour of the attachment portion of the intracorporeal device. In embodiments, the recess may have an inner contour with dimensions greater than those of the outer contour of the attachment portion of the intracorporeal device, so that the attachment portion is retained in the recess by friction.

In embodiments, first means for coupling with the intracorporeal device and/or second means for releasing the intracorporeal device comprises a cover configured to cover the recess. The cover may be a component of first means for coupling with the intracorporeal device, by trapping the intracorporeal device between the recess and the cover. The cover may be a component of the second means for releasing the intracorporeal device, and the intracorporeal device may be released from the recess by uncovering the recess and/or movement of the arm.

In embodiments, the cover is slidable or pivotable relative to the paddle. The cover may be slidable relative to the paddle and/or to the arm. Alternatively, the cover may be pivotable relative to the paddle and/or to the arm.

The delivery system may comprise additional or alternative means for coupling with the intracorporeal device and/or additional or alternative means for releasing the intracorporeal device.

In embodiments, first means for coupling with the intracorporeal device comprises a first paddle having at least one recess configured to receive an attachment portion of the intracorporeal device, and wherein first means for coupling with the intracorporeal device and/or second means for releasing the intracorporeal device comprises a second paddle configured to cover the recess.

The first paddle and the second paddle may be movable relative to each other, in order to conceal the recess (and secure the intracorporeal device) or reveal the recess (and release the intracorporeal device). For example, the paddles may be slidable or pivotable relative to each other. In embodiments, the first and/or second paddles are pivotably or slidably coupled to an arm of the delivery system.

In embodiments, one or more arms of the delivery device further comprise a shield configured to shield patient's tissues from the anchoring means of the intracorporeal device. This feature is particularly advantageous when the anchoring portion of the anchoring means is not received in the connector or in the recess of the first means for coupling the intracorporeal implant device. When the anchoring portion of the anchoring means extends beyond the connector (formed by first coupling means and second coupling means), it may contact and injure the patient's tissues during the delivery, positioning and/or implantation process. The shield protects the patient's tissues until the intracorporeal implant device is ready for release and anchoring.

In embodiments, the shield is slidable relative to the arm. Alternatively or additionally, the shield may be slidable relative to the paddle(s). Within the context of the present disclosure, slidable components (e.g., cover, paddle, shield, arms) are preferred to pivotable components in terms of space management. Indeed, such configurations allows to maintain the low profile required for transcatheter delivery and for patient safety. The delivery device has a low profile which allows access through narrow vessels and compartments of the patient's vasculature.

In embodiments, the shield is coupled to the of the paddle, cover, or arm. Advantageously, the shield is coupled to an outer surface of a paddle, cover, or arm to shield patient's tissues from the anchoring means of the intracorporeal device. In other words, in use, the shield is positioned between the anchoring means of the intracorporeal device and patient's tissues (such as the wall of the vessel(s) through which the intracorporeal device travels or the anatomy adjacent or surrounding the target implantation site).

In embodiments, the arm(s) comprise one or more articulations. In embodiments, the arm(s) comprise two or more elongate members which are pivotable relative to each other. The first proximal member may be directly coupled, for example pivotably coupled, to the shaft of the delivery device. The second distal member may be coupled, for example pivotably coupled, to the first proximal member. The position and configuration of each arm may be adjusted and/or controlled individually. Alternatively, the position and configuration of a group of arms or of all the arms may be adjusted and controlled together.

In embodiments, the arms may be movable from a first configuration to a second configuration. The first configuration may be a folded configuration, that is, a low profile configuration in which the arms are folded close to the shaft. In this first configuration, the delivery device may be inserted into the lumen of a delivery sheath, or directly into the vasculature of the patient, and pushed to the target implantation site. The second configuration may be an expanded configuration, that is, an implantation position, in which the arms are moved radially away from the shaft.

Adjustment and control of distal components of the delivery device and system may be effected from a proximal end of the delivery device and system, for example by a medical practitioner, by means of one or more actuators and/or controller. In embodiments, the delivery device comprises one or more actuators and/or controllers, configured to actuate and/or control one or more components of the delivery device or system, including but not limited to one or more arms; one or more connectors; first means for coupling with the intracorporeal device; second means for releasing the intracorporeal device; one or more paddles; and/or one or more shields.

Within the context of the present invention, “proximal” side refers to the side closest to the medical practitioner and outside the patient; “distal” refers to the side closest to the target implantation site, for example the annulus.

In embodiments, the delivery device comprises a cover configured to surround, partially or completely, the arms of the delivery device. This feature is particularly advantageous during the delivery process, during which uncovered arms may contact and injure the patient's surrounding tissues and vessel walls. The cover may surround the arms during the delivery process and may be removed from the arms once the distal portion of the delivery system is adjacent the target implantation site. In this embodiment, it may be possible to dispense of the use of a delivery catheter; access to narrower vessels and compartments is possible.

In embodiments, the profile of the arm cover is substantially the same as the outer contour of the arms in a folded configuration. For example, the arm cover may comprise a frustoconical proximal portion and a substantially cylindrical distal portion. The length of the arm cover may be sufficient to cover the entirely of the arms, coupling means and releasing means.

In embodiments, the arm cover is a component of the delivery device. In embodiments, the arm cover is slidably coupled to the shaft or may be slidable along the shaft. In other embodiments, the arm cover may be a component separate from the delivery device, and may be provided as part of the delivery system.

According to another aspect of the present disclosure, there is provided an intracorporeal implant device for restoring an anatomical annulus from a dysfunctional configuration to a working configuration, the intracorporeal implant device comprising a continuous band forming a substantially planar central section and a plurality of crests extending radially from said central section; one or more attachment portions consisting of a crest.

In embodiments, the band is expandable from a pre-set configuration, wherein the pre-set configuration of the band corresponds to a working configuration of the annulus. Within the context of the invention, “expandable” or “expanded” means that one or more dimensions of the expanded device is greater than the one or more corresponding dimensions of the device in the pre-set configuration. The expanded band has a configuration such that it can be secured to the degenerated or dilated annulus.

An external force may be exerted (for example by the delivery device according to the present disclosure) to the band to expand from its pre-set working configuration to enable its anchoring to the dysfunctional annulus, and upon cessation of said force, the device automatically contracts or reverts back to its pre-set working configuration. This contraction mechanism facilitates the simplification and standardisation of annuloplasty procedures. The present invention is advantageous over known annuloplasty devices, which must be adjusted in situ, in the patient at the target site, by trained expert surgeons using remotely controlled tools and imaging equipment. By contrast, in the present invention, minimal intervention, estimation and adjustment by the surgeon are required, either before (e.g., during the grasping and securing phase) or after release of the device. The implant device does not require extensive manipulation and adjustment in situ. Once delivered to the target area, the device is positioned, and the band expanded to reach the attachment sites of the dilated annulus. Upon release, the expanded band collapses, contracts or reverts to its pre-set configuration, thereby automatically contracting the annulus into its working configuration to prevent significant amounts of blood flowing back through the heart valve.

The pre-set configuration is one which restores a dysfunctional annulus into a working annulus. While sizes may differ from patient to patient, the overall average three-dimensional shape of working heart annuli is similar in patients and commonly known. The pre-set configuration may therefore translate into a three-dimensional shape suitable to restore the configuration of one or more sections of a dysfunctional annulus, and/or restore its overall configuration. The implant device is preferably manufactured with a pre-set configuration corresponding to the average three-dimensional shape of a working human heart valve for standardisation purposes, but may also be manufactured with modifications to treat specific defects and/or specific patient groups. The implant device may be supplied in different sizes.

Generally, “restoring” the annulus refers to the reduction of one or more dimensions of a pathological (large) annulus. Whilst the invention seeks to restore the annulus from a dysfunctional or degenerated configuration to a working configuration, it is envisaged to use the implant device and method in the context of preventative and palliative procedures, where it is desired to maintain or preserve one or more dimensions or shape of the annulus.

In embodiments, the band is compressible from the pre-set configuration. Within the context of the invention, “compressible” or “compressed” means that one or more dimensions of the compressed device is smaller than the one or more corresponding dimensions of the device in the pre-set configuration. The band may be deformable, foldable or collapsible, so as to achieve an expanded configuration and/or a compressed configuration. The compressed band has a configuration such that it can be fitted in the inner lumen of a delivery catheter or delivery sheath. Preferably, the compressed band is slidably fitted in the inner lumen of a delivery catheter or delivery sheath.

Transcatheter procedures are minimally invasive and allow patients to experience less discomfort and to recover in a shorter amount of time, compared to surgical procedures. However, transcatheter procedures involve smaller devices and instruments, which are more difficult to manipulate, and the surgeon's movement is further limited by hindrance and obstacles in the patient's own anatomy. Access is not an issue with surgical procedures, and there is therefore little need for additional complex insertion, delivery, viewing and implantation instruments. The implant device according to the present disclosure may be both expandable and compressible, and requires minimal manipulation in situ so that simple transcatheter instruments may be used.

The overall (or some) dimensions of the annulus in a working configuration are the same or smaller than the annulus in a degenerated configuration. Therefore, in embodiments, the implant device has a pre-set configuration with overall (or some) dimensions which are smaller than the dimensions of the expanded device in the deployed configuration; and/or the implant device has a pre-set configuration with overall (or some) dimensions which are greater than the dimensions of the collapsed or compressed device in the delivery configuration. In embodiments, the dimensions of the band in the expanded configuration are equal or greater than the dimensions of the degenerated annulus. Thus, the band can be expanded so as to reach the attachment sites of the annulus, and secure the band thereto.

In embodiments, the band, in its pre-set configuration, is substantially rigid. The pre-set configuration of the band is a configuration which supports the annulus in a working configuration. Since the working configuration is the configuration in which the backwards flow is significantly minimised, there may be more than one working configuration of the annulus. The band in its pre-set configuration may be sufficiently rigid to act as a support, scaffolding or skeleton for the annulus. When the band is secured in its pre-set configuration to the annulus, it does not only narrow the circumference of the dilated annulus, but it also re-shapes the annulus.

A notable difference from known implants lies in the attachment portion of the intracorporeal implant device. The attachment portion interfaces with first coupling means and second releasing means of the delivery device. In the present disclosure, an attachment portion may consist of a crest (or of the semi-elliptical or uppermost portion of a crest) and optionally of the anchoring means (or part of the anchoring means). The intracorporeal implant device may be devoid of any additional attachment means. The implant design is simple and easy to manufacture, whilst being efficiently and safely coupled and released from the delivery device.

In embodiments, the intracorporeal implant device is crown-shaped with a peripheral palisade of crests. In embodiments, each crest may be formed of a semi-elliptical section of the band. The vertex of the crest(s) may be round-ended or flat-ended.

Thus, in embodiments, the one or more attachment portions of the intracorporeal implant device consist of a crest and, optionally, of an anchoring means. More specifically, an attachment portion may consist of one crest (or part thereof) and of the anchoring means (or part thereof) stemming therefrom.

In embodiments, each crest of the intracorporeal implant device comprises an anchoring means.

In embodiments, one or more crests are devoid of an anchoring means. The intracorporeal implant device may comprise a pattern of anchoring means, from which one or more anchoring means are absent in certain positions, such that, when implanted, the implant device has minimal or no contact with the heart's conduction system. The cardiac conduction system comprises a network of nodes (e.g., AV nodes), specialised cells (e.g., conducting cells and muscle cells) and sends signals to control heartbeats. The cardiac conduction system sends signals which force different parts of the heart to relax and contract. An advantage of avoiding anchoring in the heart conduction system is a safer and more secure anchoring. Indeed, when the implant is anchored in regions forming part of the heart conduction system, the anchored implant is likely to bounce with each heartbeat, hence increasing the risk of the implant becoming accidentally detached from the annulus. Such accidental detachment will cause tissue trauma, in addition to the initial trauma of anchoring an implant in key regions of the heart regulating heartbeats.

Directional terms as used herein—for example up, down, right, left, front, back, top, bottom, distal, and proximal—are made only with reference to the figures as drawn and are not intended to imply absolute orientation.

In embodiments, the anchoring means extend inside or below the crest. The anchoring means may extend from the crest, for example from the inner side of the vertex of the crest and for example towards the inside of the crest shape.

In embodiments, the anchoring means comprises one or more pins. Pins are preferred to for example barbs or screws because barbs or screws tend to dig a hole about themselves when entering the patient's tissues so that they tend to be less securely retained. By contrast, pins are less traumatic and are retained due to the inward force the of the expandable implant device. In embodiments, the anchoring means comprises or consists of one or more substantially straight pins. In embodiments, the anchoring means comprises or consists of one or more curved pins.

In embodiments, the anchoring means (e.g., a pin) extends within the plane formed by the crest. That is, the angle formed by the plane of the crest and the anchoring means is substantially 0°. In embodiments, the anchoring means extends at an angle from the plane formed by the crest. The anchoring means may form an angle α of 135° or 180° relative to the plane formed by the crest. The angle α may be comprised between 90° and 180°, preferably between 135° and 180°. The value of the angle α depends on the amount of expansion required to provide optimal entry into the tissue and optimum grasping of the tissue.

In embodiments, the intracorporeal implant device is integrally formed. The implant device may be made of a resilient material, such polymers, co-polymers, or shape-memory metal, such as nitinol.

According to another aspect of the present disclosure, there is provided a system for the delivery and securing of an intracorporeal device to a target site in a patient, wherein the system comprises a delivery device according to the present disclosure and an intracorporeal implant device. In embodiments, the intracorporeal implant device is an intracorporeal implant device according to the present disclosure. Other intracorporeal implant device may be delivered using the delivery device according to the present disclosure.

In embodiments, the delivery system further comprises an annulus positioner. The positioner is configured to modify and support the annulus prior to and during the implantation process, in a configuration suitable to receive the intracorporeal implant device. The positioner is a component configured to prepare the annulus prior to implantation of the intracorporeal device. The annulus may be shaped and positioned to receive the intracorporeal device, before the intracorporeal device is released from the delivery device.

The positioner may be provided as a separate tool, which may be coupled to one or more components of the delivery system or simply used in combination with a delivery system as described in the present disclosure. Alternatively, the positioner may be coupled or form part of the delivery device according to the present disclosure. In embodiments, the annulus positioner is coupled to the delivery device. For example, the annulus positioner may be coupled to or along he shaft of the delivery device. In embodiments, the annulus positioner (or the expandable cage) may be slidably coupled to the shaft of the delivery device.

In embodiments, the positioner comprises an expandable cage, which may be self-expandable or controllably expandable. In the expanded state, an outer contour of the positioner is in contact with and/or exerts pressure to an inner contour of the annulus. In a passive reconfiguration step, the positioner may act as a stabilizer by substantially preventing movement of the annulus during the implantation of the implant device. In an active reconfiguration step, the positioner may be used to adjust and/or hold the annulus in a desired configuration. Accordingly, the dilated or degenerated annulus may be arranged and stabilized in a receiving configuration to receive the annuloplasty device.

In embodiments, the expandable cage may be substantially spherical, ovoid, hourglass-shaped, conical, or bell-shaped. The cage may be open or closed.

In embodiments, the expandable structure comprises or consists of a shape memory material, such as nitinol, and/or resilient components, such as springs.

In embodiments, the positioning device comprises or has a symmetrical outer contour. This symmetry distributes the pressure exerted by the positioning device homogeneously to the annulus, thereby minimising the risk of tissue trauma or injury.

In embodiments, the cage comprises a mesh and/or cover. However, open structures are preferred to allow blood flow during the delivery and/or implantation procedure. In addition, unencumbered structures are preferred to avoid hindrance and entanglement during the annuloplasty device implantation process.

According to another aspect of the present disclosure, there is provided a method for restoring an anatomical annulus of a patient from a dysfunctional configuration to a working configuration, the method comprising the steps of: delivering an intracorporeal implant device for restoring an anatomical annulus from a dysfunctional configuration to a working configuration using a delivery device according to the present disclosure; positioning the intracorporal implant device; and releasing the intracorporal implant device.

In embodiments, the method comprises the step of coupling the intracorporeal implant device to the delivery device by means of first means for coupling with the intracorporeal device.

In embodiments, the method comprises the step of shielding patient's tissues from the anchoring means of the intracorporeal device during delivery, by means of a shield.

In embodiments, the method comprises the step of shielding patient's tissues from the arms of the delivery device during delivery, by means of a cover configured to surround the arms of the delivery device.

In embodiments, the method comprises the step of modifying the shape of the annulus by means of an annulus positioner.

In embodiments, the method comprises the step of adjusting the anchoring angle of the anchoring means.

In embodiments, the method comprises the step of releasing the intracorporeal implant device from the delivery device by means of second means for releasing the intracorporeal device.

In embodiments, the incorporeal implant device is an incorporeal implant device according to the present disclosure.

In embodiments, the method is a transcatheter method.

In embodiments, the anatomical annulus is a heart valve annulus. The heart valve may be the tricuspid valve. The annulus may be the anterior annulus, the posterior annulus and/or the septal annulus. The heart valve may be the mitral valve, and the annulus may be the anterior annulus, the posterior annulus, the lateral commissure and/or the medial commissure. Most annuloplasty procedures are directed at reducing the posterior annulus. However, the portions between the anterior and posterior annulus (the commissures) are also known to elongate over time and can be reshaped using a device according to the present invention.

### ACCOMPANYING FIGURES

The details of one or more implementations are set forth, by way of example only, in the accompanying drawings and the description below. Other features, aspects, and advantages of the disclosure will become apparent from the description, the drawings, and the claims, in which:

FIG. 1 is a schematic (top view) representation of an intracorporeal implant device according to the present disclosure;

FIGS. 2A and 2B are schematic (top perspective view and side view, respectively) representations of the intracorporeal implant device of FIG. 1;

FIGS. 3A and 3B are schematic (top perspective view and side view, respectively) representations of another intracorporeal implant device according to the present disclosure;

FIGS. 4A and 4B are schematic (top perspective view and side view, respectively) representations of another intracorporeal implant device according to the present disclosure;

FIGS. 5A and 5B are schematic (top perspective view and side view, respectively) representations of another intracorporeal implant device according to the present disclosure;

FIGS. 6 to 9 are a schematic representations of a delivery system according to the present disclosure, in different configurations;

FIGS. 10 to 12 are partial schematic representations of a delivery device according to the present disclosure, in different configurations; and

FIGS. 13 to 16 are partial schematic representations of a delivery device according to the present disclosure, in different configurations.

The embodiments described herein are provided as exemplary and non-limiting embodiments of the present invention.

### DETAILED DESCRIPTION

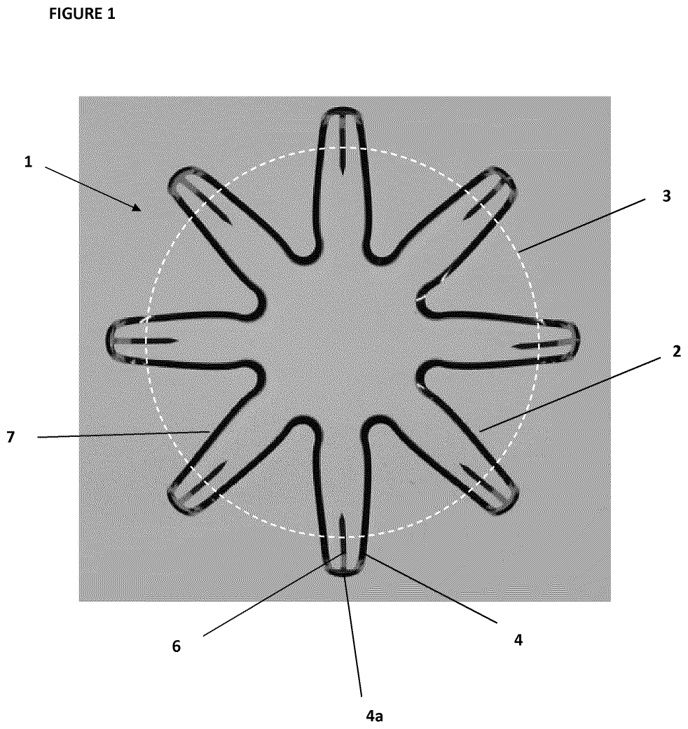

With reference to FIG. 1, there is illustrated an intracorporeal implant device 1 for restoring an anatomical annulus from a dysfunctional configuration to a working configuration, the intracorporeal implant device 1 comprising a continuous band 2 forming a central section 3 and a plurality of crests 4 extending radially from said central section 3; one or more attachment portions 5 consisting of a crest 4 and, in this example, of an anchoring means 6.

Within the context of the present disclosure, the annulus may have a “dysfunctional configuration” in which it has partially or completely lost its functional activity, namely, to provide structural support to the valve so that the valve allows flow from a first compartment to a second compartment and minimises backflow from the second compartment to the first compartment. A dysfunctional annulus may be (genetically or pathologically) degenerated and the terms are used herein interchangeably. The degeneration may for example originate from injury, malformation, or injury, and often expresses itself as a loosening or dilatation of the annulus. By contrast, a “working configuration” is a configuration wherein the annulus provides adequate and sufficient structural support so as to minimise or avoid valve regurgitation.

In FIG. 1, the implant device 1 is integrally formed with a shape-memory material, such as nitinol. The implant device 1 is crown-shaped or flower-shaped.

The implant device 1 comprises a central portion, which may be substantially planar or may be concave to follow the contours of an anatomical annulus. The band 2 may curve towards the centre of the central portion 3, thereby forming an inner portion unhindered by the implant device which may be aligned with the centre of the patient's annulus. The implant device 1 may comprise a plurality of arms 7. The arms 7 may extend into or form the central portion 3 of the implant device 1. The arms 7 may form crests 4 extending radially from the central portion 3.

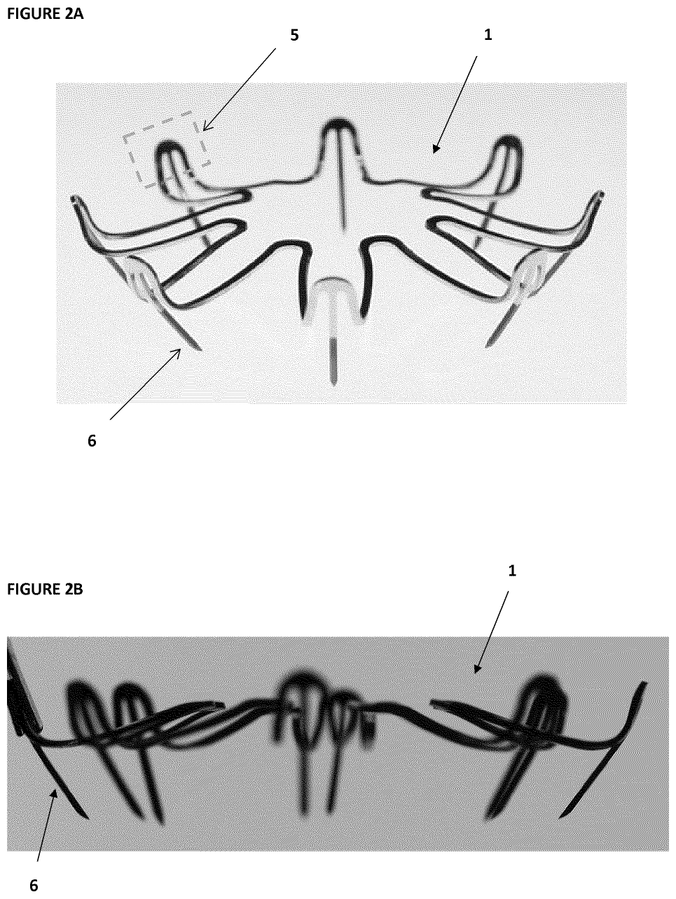

As illustrated in FIGS. 2A and 2B, the crests 4 are shown to extend upwards from the central portion 3. In embodiments, the crests 4 may extend from the central portion 3 at an angle of from around 90° to around 45°, preferably around 90°.

In embodiments, the anchoring means are pins 6. The anchoring means 6 stems from the band 2, more preferably from a crest 4, most preferably from the vertex 4a of the crest 4. In preferred embodiments, the anchoring means 6 extend inwards of the crest 4.

In embodiments, the anchoring means 6 have a first end connected to the band 2 and a second free end for anchoring in the patient's tissues. The second free end preferably points inwards of the implant device 1. Each anchoring means or pin 6 may be constituted of an attachment segment extending radially from the central portion 3 of the implant device 1, in the direction of the crests 4, and of an anchoring segment extending radially from the central portion 3 of the implant device 1, in the direction opposite of that of the crests 4.

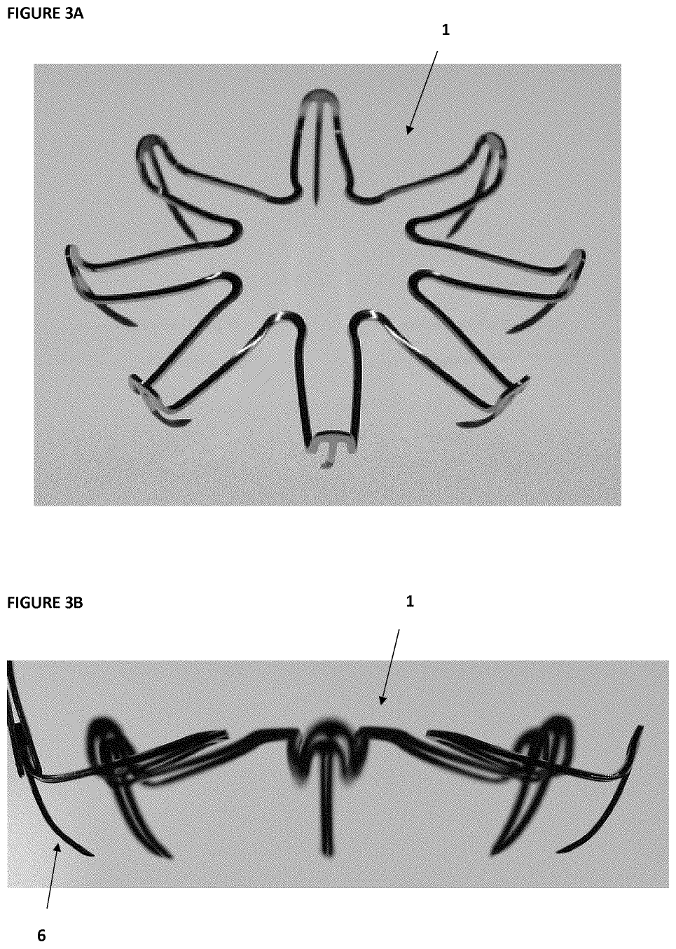

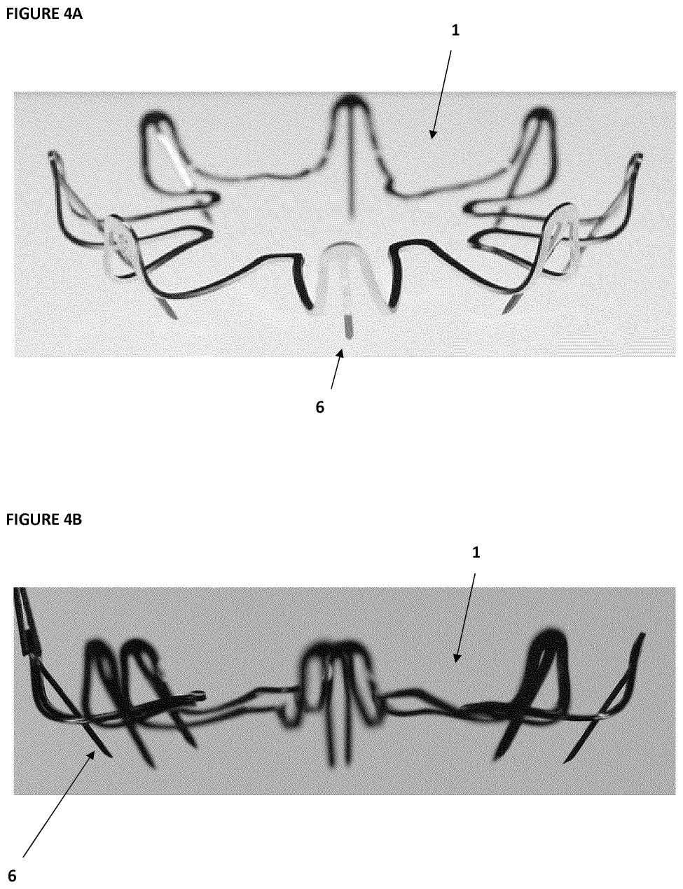

The anchoring means 6 may comprise one or more bends, as illustrated in FIGS. 2A and 2B. The anchoring means 6 may be curved, as illustrated in FIGS. 3A and 3B. The anchoring means 6 may be substantially linear, as illustrated in FIGS. 4A and 4B.

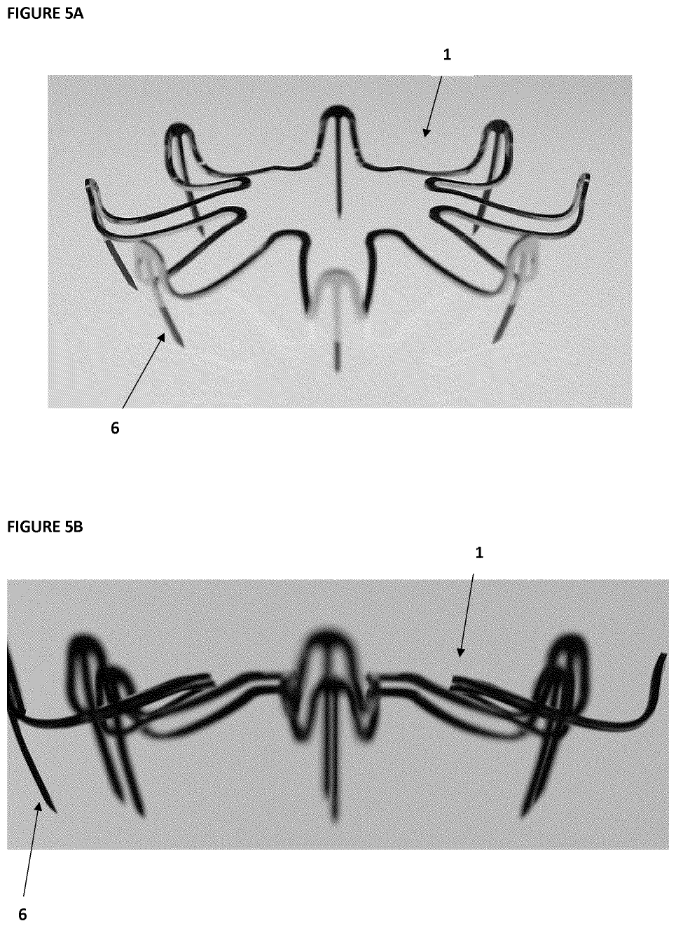

In embodiments, an anchoring means 6 is substantially coplanar with the crest 4 it is coupled to, or comprise a portion which is substantially coplanar with the crest 4 as illustrated in FIGS. 5A and 5B. That is, the angle formed by the plane of the crest and the anchoring means is substantially 0°. In embodiments, the anchoring means extends at an angle from the plane formed by the crest. The anchoring means may form an angle a of 135° or 180° relative to the plane formed by the crest. The angle a may be comprised between 90° and 180°, preferably between 135° and 180°. The value of the angle a depends on the amount of expansion required to provide optimal entry into the tissue and optimum grasping of the tissue.

The attachment portion 5 consists of the crest 6 (or of the distal portion of the crest 4 relative to the central portion 3) and optionally of the anchoring means 6 (or of the distal portion of the anchoring means 6 relative to the central portion 3). An exemplary attachment portion 5 is illustrated in FIG. 2A, which consists of the distal portion of the crest 4 and of the distal portion of the anchoring means 6.

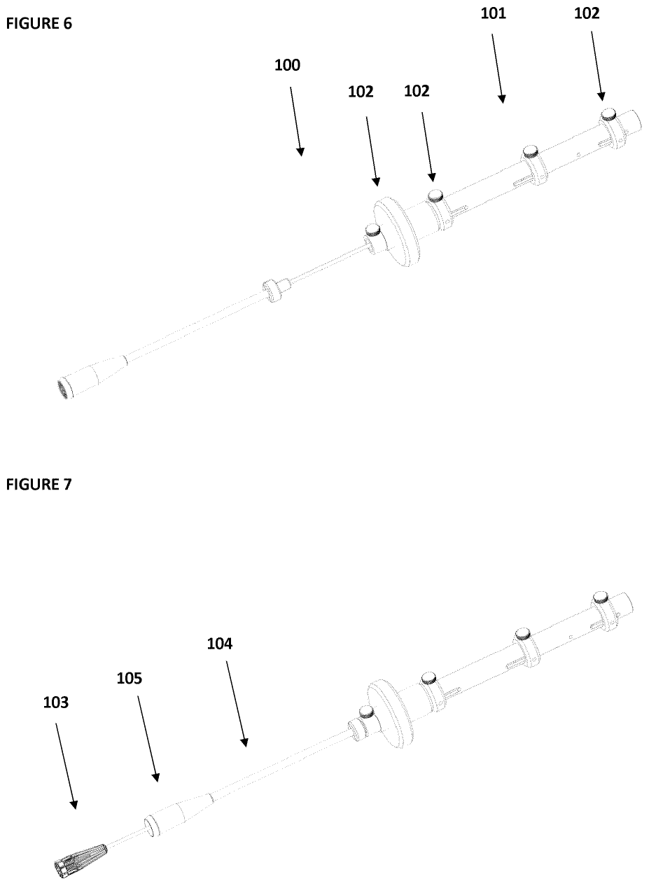

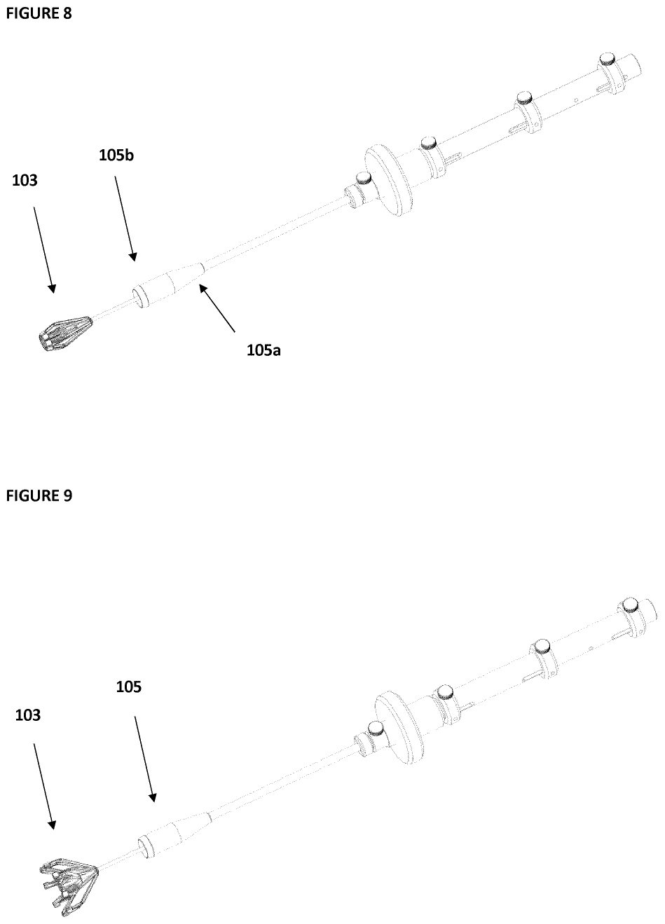

FIGS. 6 to 9 illustrate a delivery system 100 according to the present disclosure. The delivery system 100 may comprise a handle 101 for the medical practitioner to manipulate the components of the delivery system 100. The handle 101 may comprise a number of actuators 102 to manipulate, control and/or adjust one or more components of the delivery system 100. The actuators 102 may comprise a push-button and/or rotatable controllers.

The delivery system 100 comprises a delivery device 103 adjacent or at the distal end thereof. The delivery device 103 may be coupled to the handle by means of a shaft 104. The shaft 104 is configured to navigate through the patient's anatomy and more particularly vasculature. The shaft 104 is preferably flexible, or comprises flexible portions, for example a combination of rigid or semi-rigid portions and flexible portions. The movement and configuration of the shaft 104 may be controlled by one or more actuators 102.

The delivery system 100 may comprise a cover 105 configured to surround the arms 106 of the delivery device 103. The cover 105 may comprise a substantially cylindrical portion 105b, or a frusto-conical portion 105a and a substantially cylindrical portion 105b. One portion, for example, the substantially cylindrical portion 105b is configured to surround the distal portion or member 106b of the arms. One portion, for example, the frusto-conical portion 105a, is configured to surround the proximal portion or member 106b of the arms. The cover 105 protects the patient's tissues against injury which may be caused by the delivery device 103, and more particularly the arms 106, when the delivery device 106 navigates through the patient's anatomy. The cover 105 also secures the delivery device 103 into a folded configuration. Movements of the cover 112 may be controlled by one or more actuators 102.

The arms 106 of the delivery device 103 may comprise one or more articulations 107. An articulation 107 may separate two members 106a, 106b of an arm 106. The arms 106 may move between a first folded configuration and a second expanded configuration. The movement and configuration of the arms 106 may be controlled by one or more actuators 102.

The movement between the first and the second configurations may be effected through a linear or longitudinal motion of the shaft 104 relative to the handle 101 and/or the delivery device 103. For example, the shaft 1 may be coupled to an actuation rod 108 configured to convert the longitudinal motion of the shaft 104 into a radial movement of the arms 106. The actuation rod 108 is slidably received in a lumen of shaft 104, and the distal end of the actuation rod 108 extends beyond the distal opening of the lumen of the shaft 104. The actuation rod 108 may be coupled to the distal and of the shaft 104, the shaft 104 may act as an actuation rod. Displacement of the actuation rod 108 may be controlled by one or more actuators 102.

The actuation rod 108 may be coupled to a plurality of stretchers 109 configured to move the arms 106 between the first and the second configurations. The stretchers 109 may be coupled to the actuation rod 108 and to the distal end or distal portion of the arms 106.

FIGS. 13 to 16 illustrate further features of the delivery device 103. The delivery device 103 may first means for coupling with the intracorporeal device 1 and second means for releasing the intracorporeal device 1.

First means for coupling with the intracorporeal device 1 may be coupled to or adjacent the distal end of the arm 106. Coupling means may comprise a paddle 110 having a recess 111. The recess 111 may be formed in any other support member capable of having a recess 111 formed therein. The recess 111 is configured to receive an attachment portion 5 of an intracorporeal device 1. In the embodiment illustrated in FIGS. 13 to 15, the vertex portion of the crest 4 and the attachment segment of the anchoring means 6 constitute the attachment portion 5. Nonetheless, the recess 111 may be configured to receive and secure any attachment portion of the intracorporeal device 1. The recess 111 may be shaped and dimensioned to receive the attachment portion 5 of the intracorporeal device. The recess 111 may have a profile substantially matching that of the attachment portion 5. The attachment portion 5 may be secured in the recess by friction and/or by means described hereinbelow.

The delivery device 103 may comprise a cover 112 configured to cover the recess 111. In the illustrated embodiments, the cover 112 is slidable relative to paddle 110 between a first position in which the implant 1 is secured to the delivery device 103 and a second position in which the implant 2 may be released. In an alternative embodiment, the cover 112 may be pivotable relative to the paddle 110 between a first position in which the implant 1 is secured to the delivery device 103 and a second position in which the implant 2 may be released. Displacement of the cover 112 may be controlled by one or more actuators 102.

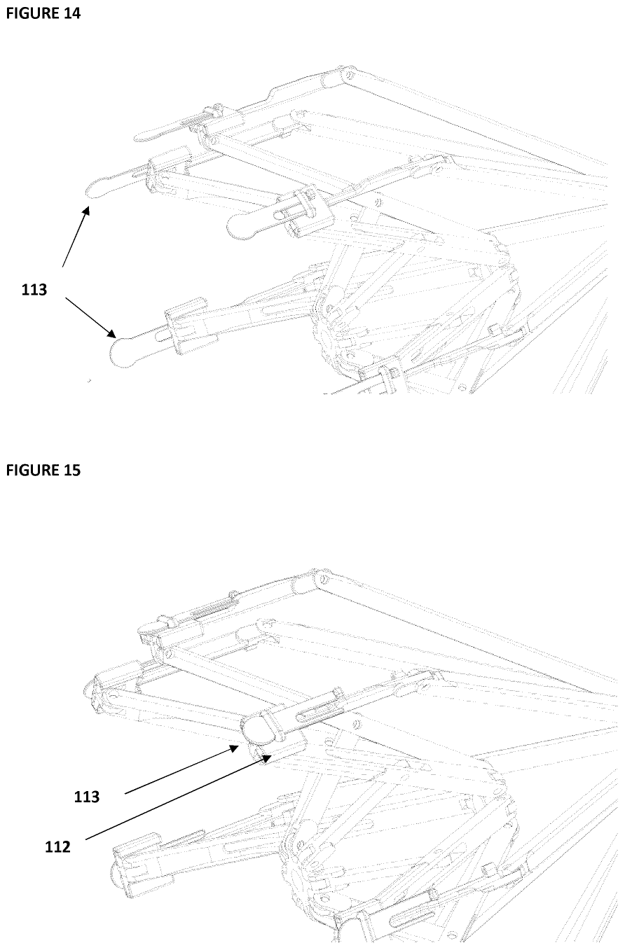

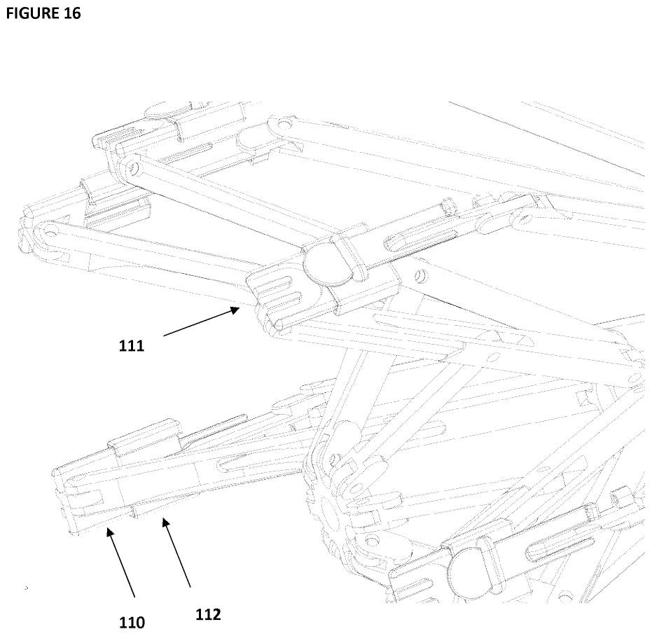

The delivery device 103 may comprise a shield 113. The shield 113 is configured to shield patient tissues from the anchoring means 6 of the intracorporeal device 1. The shield 113 may be configured to be displaced between a first shielding configuration in which the anchoring means 6 is covered by the shield 113 (as illustrated for example in FIGS. 13 and 14) and a second anchoring configuration in which the anchoring means 6 is uncovered (as illustrated for example in FIGS. 15 and 16). In the first shielding configuration, the shield 113 is positioned between the anchoring means 6 and patient's tissues and prevents potential injury during the delivery process. The shield 113 may be displaced into the anchoring position 113 when the delivery device 103 is positioned adjacent the target implantation site.

The shield 113 may be coupled to the arm 106 of the delivery device 103, to the cover 105 and/or to the paddle 110. Displacement of the shield 113 may be controlled by one or more actuators 102.

The present disclosure also concerns methods for restoring an anatomical annulus of a patient from a dysfunctional configuration to a working configuration. The method comprises the steps of delivering an intracorporeal implant device, positioning the intracorporal implant device; and releasing the intracorporal implant device.

An exemplary method will now be outlined, using an intracorporeal device 1 according to the present disclosure and a delivery device 103 according to the present disclosure.

Prior to insertion into the patient, the intracorporeal device 1 is secured to the delivery device 103. The attachment portion 5 is inserted into a recess 111 of paddle 110 of the delivery device 103. The cover 112 slides across the paddle 110 to cover the recess 111, thereby securing the attachment portion 5 of the intracorporeal device 1 between the paddle 110 and the cover 112. The intracorporeal implant device 1 is tightly secured to the delivery device at multiple points, and the coupling means of the delivery device 103 prevent or minimise the risk of the implant 1 becoming accidentally detached. Coupling means also enables the safe and accurate handling of the intracorporeal device 1. The accuracy may further be enhanced where the configuration of each arm 106 is individually adjustable.

The shield 113 is displaced over the anchoring means 6 into a shielding configuration, as illustrated for example in FIGS. 13 and 14. In this configuration, the attachment portion 5 of the intracorporeal device 1 is secured between the paddle 110 and the cover 112, and the anchoring portion of the intracorporeal device 1 which extends from the paddle 110 is shielded by shield 113. In the delivery configuration, the intracorporeal device 1 is fully shielded, thereby preventing the risk of injury to the patient's tissues. For example, the tip of the anchoring pin 6 is shielded such that it cannot prick or scratch the patient's anatomy.

The arms 106 of the delivery device 103 are folded into a low-profile delivery configuration, as illustrated for example in FIG. 14. The cover 105 for the delivery device 105 is displaced along the shaft 104 until the cover 105 surrounds the arms 106 of the delivery device 103, as illustrated for example in FIG. 6. In the delivery configuration, the delivery device 103 is fully covered, thereby preventing the risk of injury to the patient's tissues. No part of the delivery device 103 is exposed such that it cannot prick or scratch the patient's anatomy.

The delivery device 103 is inserted into the patient's anatomy and pushed, for example, through the patient's vascular system, until the delivery device 103 reaches the target implantation site. The configuration of the shaft 104 may be controlled and adjusted to facilitate navigation through the patient's anatomy. One or more markers may be provided on the shaft 104 (for example, marker rings), on the cover and/or on the delivery device. The delivery system 100 may comprise one or more actuators 102 at the distal end of the delivery system 100 to control and actuate one or more components of the delivery system 100. The actuators 102 are advantageously located at the proximal end portion of the delivery system 100, for example on or in the handle 101. The delivery system 100 may comprise an external controller system and/or an external imaging system to facilitate the delivery and/or implantation of the intracorporeal device 103. The controller system may be used to control one or more components of the delivery system 100. The imaging system may be used to visualise one or more components of the delivery system 100.

The configuration of the annulus may be prepared by means of a separate or integral annulus positioner (not shown). The annulus position may comprise an expandable cage to push and support the annulus in an optimum position to receive the intracorporeal device 1.

Once the delivery device 103 is adjacent the target implantation site, for example a heart valve annulus, the position and orientation of the delivery device 103 can firstly by adjusted by movement of the end portion of the shaft 104.

When the delivery device 103 is suitably positioned, the delivery device cover 105 may be withdrawn, thereby revealing the delivery device 103, as illustrated for example in FIG. 7.

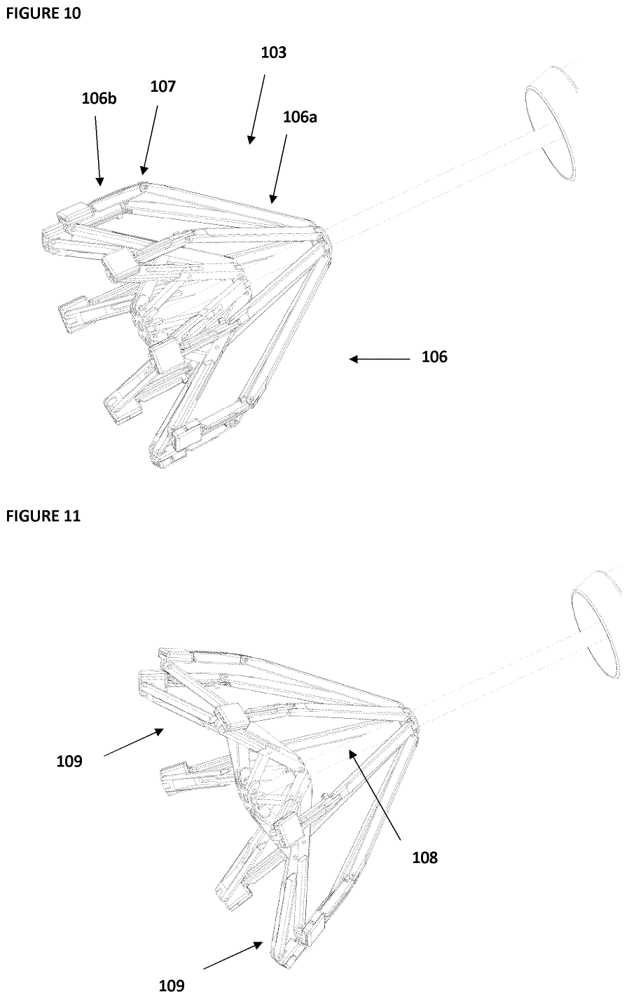

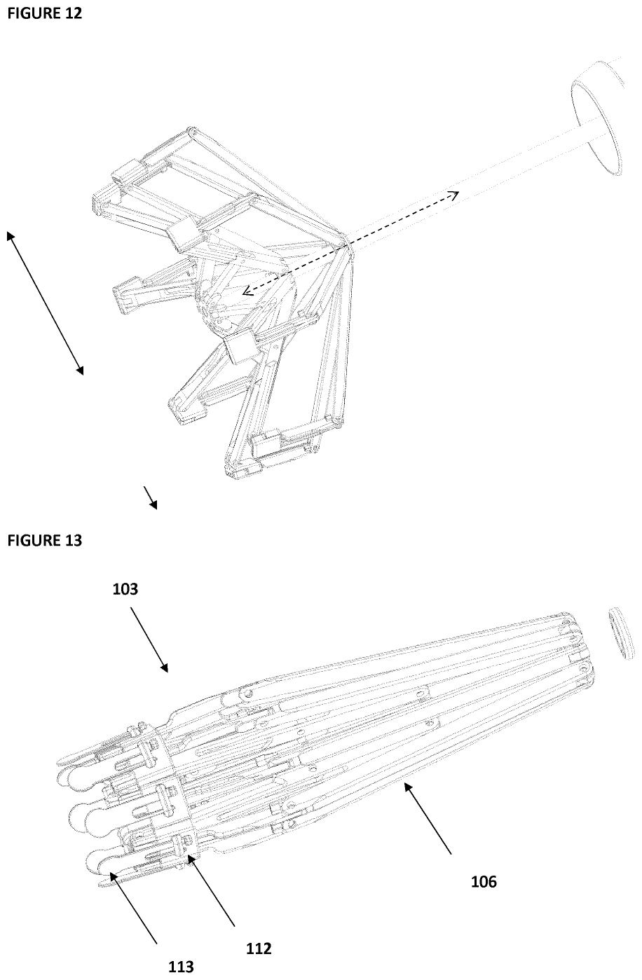

The position and orientation of the delivery device 104 are further adjusted by movements of the arms 106 of the delivery device 103. The actuation rod 108 may be displaced to control the expansion of the arms 106, as illustrated for example in FIGS. 10 to 12. The actuation rod 108 may be pushed in a distal direction. The stretchers 109 straighten to displace the arms 106 radially outward relative to the shaft 104 or to the actuation rod 108.

When the overall position and orientation of the delivery device 103 is optimal, the overall configuration of the intracorporeal device 1 and the orientation of the anchoring means 6 may be adjusted.

The distal member 106b of the arms 106 may be displaced to adjust the overall configuration of the intracorporeal device 1. The intracorporeal device 1 is expanded from its pre-set configuration to a dimensions greater than that of the degenerated annulus. Advantageously, the intracorporeal device 1 is positioned and oriented such that areas of the intracorporeal device 1 devoid of anchoring means 6 faces areas of the annulus which should not be implanted, such as areas, nodes or cells of the heart conduction system.

The distal member 106b of the arms 106 may be pivoted to adjust the orientation of the anchoring means 6. Advantageously, the arms 106, or distal member 106b thereof, are pivoted outward such that the anchoring portion of the anchoring means 6 (for example the tip of the pins 6) are oriented outwardly.

The shield 113 may comprise an imaging marker to visualise the orientation of the anchoring means 6. The shield 113 may be displaced to uncover the anchoring portion of the anchoring means 6 during the pin orientation process or when the intracorporeal device 1 is close-to-ready or ready for release. The shield 113 may be displaced to uncover the anchoring portion of the anchoring means 6 partially or completely, as illustrated for example in FIGS. 15 and 16. The anchoring portion of the anchoring means 6 may be positioned against the target anchoring points in the annulus.

Once the intracorporeal device 1 is ready for release, the cover 112 is displaced to reveal the attachment portion 5 of the anchoring means 6. The displacement 112 of the cover may trigger the release of the anchoring means 6. Additionally or alternatively, the delivery device 103 may comprise further means for releasing the anchoring means 6 from the delivery device 103, such as a release pin or tab.

Upon release, the intracorporeal device 1 will automatically and simultaneously revert to its pre-set configuration, which corresponds to a working configuration of the annulus, and anchor itself to the annulus.

A problem arising from conventional annuloplasty devices is that their implantation is difficult to reverse. In other words, the devices cannot be easily removed and repositioned in the event of an unsuccessful implantation. In the present invention, anchoring means 6 are secured to the annulus by pressure from the intracorporeal device 1 (owing to its pre-configuration) may be used, so that should the intracorporeal device 1 require repositioning, the expansion of the intracorporeal device 1 would release the anchoring means 6. The intracorporeal device 1 can be repositioned with minimal trauma to the annulus.

Upon successful release and implantation of the intracorporeal device 1, the delivery device 103 may be removed. The arms 106 may be folded by pulling proximally the actuation rod 108. The delivery device cover 105 may be displaced distally to cover the delivery device 103. The delivery device 103 can then be safely pulled back and removed from the patient.

Although the present invention has been described within the context of heart valves and in particular of annuloplasty procedures, it is envisaged that it could have other advantageous implementations involving the reshaping of an anatomical structure.

The present invention is particularly beneficial in the context of transcatheter procedures and/or in the cardiovascular field. For example, in transcatheter procedures, access points are formed through tissues and/or blood vessels. The annuloplasty device may be used to restore blood vessels to their working configuration by partially or completely closing the access point(s) so as to minimise or prevent blood flow to the outside of the blood vessels.

The present invention may be used in the context of an atrial septal defect (ASD), which is a hole in the atrial septum. The ASD may be congenital, or may be the desired result of a medical procedure intended to relieve the pressure between the two atrial chambers of the heart. The implant device may be used to close the hole partially or completely so as to minimise or prevent blood flow between the atrial chambers.

Importantly, the present invention provides systems, devices and methods specifically adapted to the expedient delivery and accurate implantation of an intracorporeal implant device. Most advantageously, this is achieved whilst ensuring optimum patient safety.

### REFERENCE NUMERALS

1 Intracorporeal device, intracorporeal implant device or implant
2 Band
3 Central portion of the intracorporeal implant device
4 Crest
4a Vertex of crest
5 Attachment portion
6 Anchoring means (e.g., pin)
7 Arms
100 Delivery system
101 Handle
102 Actuator
103 Delivery device
104 Shaft
105 Cover for delivery device
106 Arm of the delivery device
106a First (proximal) member of arm
106b Second (distal) member of arm
107 Arm articulation
108 Actuation rod
109 Stretcher
110 Paddle
111 Recess
112 Cover for the recess
113 Shield

1. A device for the delivery and securing of an intracorporeal device to a target site in a patient,
wherein the delivery device comprises a shaft and a plurality of arms coupled to said shaft and configured to expand radially outwards relative to said shaft,
wherein each arm comprises first means for coupling with the intracorporeal device and second means for releasing the intracorporeal device.

2. The delivery device according to claim 1, wherein first means for coupling with the intracorporeal device comprises a paddle having at least one recess configured to receive an attachment portion of the intracorporeal device.

3. The delivery device according to claim 2, wherein the recess is configured to couple with the intracorporeal device by friction.

4. The delivery device according to claim 2, wherein first means for coupling with the intracorporeal device and/or second means for releasing the intracorporeal device comprises a cover configured to cover the recess.

5. The delivery device according to claim 4, wherein the cover is slidable or pivotable relative to the paddle.

6. The delivery device according to claim 1, wherein first means for coupling with the intracorporeal device comprises a first paddle having at least one recess configured to receive an attachment portion of the intracorporeal device, and wherein first means for coupling with the intracorporeal device and/or second means for releasing the intracorporeal device comprises a second paddle configured to cover the recess.

7. The delivery device according to claim 6, wherein the first and/or second paddles are pivotably or slidably coupled to an arm of the delivery system.

8. The delivery device according to claim 1, wherein one or more arms further comprise a shield configured to shield patient's tissues from the anchoring means of the intracorporeal device.

9. The delivery device according to claim 6, wherein the shield is slidable relative to the arm.

10. The delivery device according to claim 8, wherein the shield is coupled to the of the paddle, cover or arm.

11. The delivery device according to claim 1, wherein the arm(s) comprise one or more articulations.

12. The delivery device according to claim 1, wherein the delivery device comprises a cover configured to surround the arms of the delivery device.

13. The delivery device according to any claim 12, wherein the arm cover is slidably coupled to the shaft.

14. An intracorporeal implant device for restoring an anatomical annulus from a dysfunctional configuration to a working configuration, the intracorporeal implant device comprising:
a continuous band forming a central section and a plurality of crests extending radially from said central section;
one or more attachment portions consisting of a crest.

15. The intracorporeal implant device according to claim 14, wherein the one or more attachment portions consist of a crest and of an anchoring means.

16. The intracorporeal implant device according to claim 14, wherein each crest comprises an anchoring means.

17. The annuloplasty device according to claims 14, wherein one or more crests are devoid of an anchoring means.

18. The intracorporeal implant device according to claim 14, wherein the anchoring means extend inside or below the crest.

19. The intracorporeal implant device according to claim 14, wherein the anchoring means comprises or consists of a substantially straight pin.

20. The intracorporeal implant device according to claim 14, wherein the anchoring means comprises or consists of a curved pin.

21. The intracorporeal implant device according to claim 14, wherein the anchoring means extends within the plane formed by the crest.

22. The intracorporeal implant device according to claim 18, wherein the anchoring means extends at an angle from the plane formed by the crest.

23. A system for the delivery and securing of an intracorporeal device to a target site in a patient, wherein the system comprises a delivery device according to claim 1 and an intracorporeal implant device.

24. The system according to claim 23, wherein the intracorporeal implant device is an intracorporeal implant device for restoring an anatomical annulus from a dysfunctional configuration to a working configuration, the intracorporeal implant device comprising:
a continuous band forming a central section and a plurality of crests extending radially from said central section and one or more attachment portions consisting of a crest.

25. The delivery system according to claim 23, wherein the delivery system comprises one or more actuators and/or controllers, configured:
to actuate and/or control the shaft;
to actuate and/or control one or more arms;
to actuate and/or control one or more delivery device cover;
to actuate and/or control first means for coupling with the intracorporeal device;
to actuate and/or control second means for releasing the intracorporeal device;
to actuate and/or control one or more paddles;
to actuate and/or control one or more paddle cover; and/or
to actuate and/or control the shield(s).

26. The system according to claim 23, wherein the system further comprises an annulus positioner.

27. The system according to claim 26, wherein the annulus positioner comprises an expandable cage.

28. A method for restoring an anatomical annulus of a patient from a dysfunctional configuration to a working configuration, the method comprising the steps of:
delivering an intracorporeal implant device for restoring an anatomical annulus from a dysfunctional configuration to a working configuration using a delivery device according to claim 1;
positioning the intracorporal implant device; and
releasing the intracorporal implant device.

29. The method according to claim 28, comprising the step of coupling the intracorporeal implant device to the delivery device by means of first means for coupling with the intracorporeal device.

30. The method according to claim 28, comprising the step of shielding patient's tissues from the anchoring means of the intracorporeal device during delivery, by means of a shield.

31. The method according to claim 28, comprising the step of shielding patient's tissues from the arms of the delivery device during delivery, by means of a cover configured to surround the arms of the delivery device.

32. The method according to claim 28, comprising the step of adjusting the anchoring angle of the anchoring means.

33. The method according to claim 28, comprising the step of releasing the intracorporeal implant device from the delivery device by means of second means for releasing the intracorporeal device.

34. The method according to claim 28, wherein the incorporeal implant device is an incorporeal implant for restoring an anatomical annulus from a dysfunctional configuration to a working configuration, the intracorporeal implant device comprising:
a continuous band forming a central section and a plurality of crests extending radially from said central section and one or more attachment portions consisting of a crest.

35. The method according to claim 28, wherein the method is a transcatheter method.

36. The method according to claim 28, wherein the anatomical annulus is a heart valve annulus.
